(19) United States
(12) Patent Application Publication
(10) Pub. No.: US 2016/0000692 A1
(43) Pub. Date: Jan. 7, 2016

(54) Whipped Foundation

(71) Applicants: Tatyana Zamyatin, Princeton, NJ (US); Dariush Hosseinpour, Edison, NJ (US); Sarah Jabush, Jackson, NJ (US)

(72) Inventors: Tatyana Zamyatin, Princeton, NJ (US); Dariush Hosseinpour, Edison, NJ (US); Sarah Jabush, Jackson, NJ (US)

(73) Assignee: Revlon Consumer Products Corporation, New York, NY (US)

(21) Appl. No.: 14/322,279

(22) Filed: Jul. 2, 2014

(51) Int. Cl. A61K 8/894; A61K 8/891; A61K 8/58; A61Q 1/06; A61Q 1/02; A61Q 1/10; A61Q 1/04; A61K 8/31; A61Q 1/00

(52) U.S. Cl. CPC A61K 8/894; A61K 8/31; A61K 8/891; A61K 8/585; A61Q 1/00; A61Q 1/02; A61Q 1/10; A61Q 1/04; A61Q 1/06; A61K 2800/43

(57) ABSTRACT

The present disclosure is directed to a composition having about 7.5% to about 11.0% PEG/PPG emulsifier, an at least one diluent, an at least one emollient, and <25 weight % water, wherein the composition deviates from an initial configuration after application of about 0.5 to about 5 kPa of pressure on the composition and returns to the initial configuration in less than about 4 hours at room temperature.

This application claims priority from co-pending international application number PCT/U.S.13/20028, filed Jan. 3, 2013, which claims priority to U.S. provisional application 61/583,854, filed Jan. 6, 2012, now expired. The entire disclosure of PCT/U.S.13/20028 is hereby incorporated by reference.

Cosmetic compositions, are commercially available in various forms, such as creams and mousses, and are well known in the art. These compositions are typically formulated to allow easy application and a pleasing texture. One major drawback with these compositions is the disruption of the composition surface and subsequent used look when pressure is applied to the surface of the composition.

It is desirable that a composition used as a cosmetic composition, retain a fresh unused look of an untouched surface. Such a composition would be appealing to the consumer. It has been found that the combination of PEG/PPG emulsifier, diluent, emollient, and water in the present cosmetic composition has an improved texture that retains a fresh unused look.

### SUMMARY

The present disclosure is directed to a composition comprising an about 7.5% to about 11.0% PEG/PPG emulsifier, an at least one diluent, an at least one emollient, and <25 weight % water, wherein the composition deviates from an initial configuration after application of about 0.5 to about 5 kPa of pressure on the composition and returns to the initial configuration in less than about 4 hours at room temperature.

In one embodiment the composition contains an emollient. The emollient can be a straight chain hydrocarbon or a cyclic hydrocarbon. An example of an emollient includes a <100 cst dimethicone.

In one embodiment the composition is in the form of a make-up. A makeup can be an eye shadow, a concealer, an eyeliner, a make-up for the lips, a make-up for the eyelashes, a make-up for the eyebrows, and a make-up for the face.

In an embodiment of the invention the composition contains an emulsifier such as PEG/PPG 19/19 dimethicone. The dimethicone may be present in an amount of about 60.0 weight %. In another embodiment the emulsifier can be a PEG/PPG 18/18 dimethicone.

In one embodiment of the invention the composition further comprises a non-polar solvent.

In one embodiment the composition has a stable viscosity. The stable viscosity is determined by variation from the initial viscosity. The initial viscosity is measured at about 170,000 to about 670,000 CPS.

In one embodiment the composition contains a diluent and the diluent is for example an isoparaffin, a cyclopentasiloxane, or a combination of diluents.

In an embodiment the composition contains water in a concentration of about 1.0 weight % to about 17.5 weight %. In a preferred embodiment water concentration is about 17 weight %.

In one embodiment the composition described herein includes, but is not limited to, the following optional ingredients, such as a coloring agent chosen from a dye, a pigment, natural colorants, or a combination of a dye, a pigment, and natural colorants. Additional non-limiting optional ingredients include, for example, a sunscreen, a fragrance, masking agent, flavor, sweeteners, odor absorbents, and methanol.

In one embodiment the composition is in the form of a make-up. Examples of make-up include, but are not limited to, an eye shadow, concealer, and eyeliner, a make-up for the lips, a make-up for the eyelashes, a make-up for the eyebrows, and a make-up for the face. An example of a make-up for the lips is a lipstick or a lip-gloss.

### BRIEF DESCRIPTION OF THE DRAWINGS

FIG. 1 exemplifies the recovery to the initial configuration of the claimed composition over a period less than 4 hours after application of about 0.5 to about 5 kPa pressure.

### DETAILED DESCRIPTION

The terms used in this specification generally have their ordinary meanings in the art, within the context of the invention, and in the specific context where each term is used. Certain terms are discussed below, or elsewhere in the specification, to provide additional guidance to the practitioner in describing the compounds, compositions, and methods of the invention and how to make and use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. The use of examples anywhere in this specification, including examples of any terms discussed herein, is illustrative only, and in no way limits the scope and meaning of the invention or of any exemplified term. Likewise, the invention is not limited to the examples presented.

The present disclosure is directed to a composition comprising an about 7.5% to about 11.0% PEG/PPG emulsifier, an at least one diluent, an at least one emollient, and <25 weight % water, wherein the composition deviates from an initial configuration after application of about 0.5 to about 5 kPa of pressure on the composition and returns to the initial configuration in less than about 4 hours at room temperature.

The compositions of the present invention are uniquely formulated to provide not only a stable viscosity over a broad temperature range, but an enhanced feeling of softness and silkiness when the composition is applied to the skin. Moreover, the compositions are capable of easy and uniform application over the skin. These enhanced properties are achieved, in large part, by formulating the compositions with a specific concentration of PEG/PPG emulsifier, an emollient, a diluent, and water in a concentration ratio such that the viscosity remains stable over a broad temperature range.

The term “stable viscosity”, as used herein is meant to define a composition having a viscosity that does not deviate more than about 20% from an initially measured viscosity. Preferably, the stable viscosity does not deviate more than 10% from an initially measured viscosity. More preferably, the viscosity does not deviate more than 5% from an initially measured viscosity of a composition of the present invention.

For purposes of this invention the initially measured viscosity is in a range of about 170,000 to about 670,000 CPS. The viscosity measurement was taken at standard temperature and pressure.

When the phrase “at least one” is used it means one or more different ingredients are used alone or in a combination of ingredients. One of skill in the art would know the limit on how many different ingredients can be used to give the desired effect disclosed herein.

As used herein, “about” shall generally mean within 20 percent, preferably within 10 percent, and more preferably within 5 percent of a given value or range.

As used herein, secondary, indicates use of a class of ingredient used once in the composition. A secondary emulsifier is an indication that one emulsifier is already present in the composition.

The PEG/PPG emulsifier, as used herein, assists in building a matrix in the composition. The PEG/PPG emulsifier is a combination of PEG/PPG and a diluent. The length of PEG/PPG and the diluent used is determined by the ability of the PEG/PPG/diluent combination that allows recovery of the composition as defined herein. The various PEG/PPG emulsifier combinations utilize a variety of diluents. PEG/PPG/diluent combinations are found in the Ingredient Database of Personal Care Products Council http://online.personalcarecouncil.org, and are hereby incorporated by reference.

The concentration of PEG/PPG emulsifier present in the compositions are found in a concentration of about 7.5% to about 11.0%. This concentration is based on a PEG/PPG 19/19 dimethicone or a PEG/PPG 18/18 dimethicone. The concentration will change depending on the PEG/PPG emulsifier diluent combination used to obtain a composition that recovers as defined herein.

A suitable emollient for use in the present compositions is a cyclic or straight chain hydrocarbon, and includes, but is not limited to cyclomethicone, <100 cst dimethicone, siloxane, cyclopentasiloxane or any combinations thereof. An emollient provides a softening, protective or soothing effect on the skin surface and is generally considered safe for topical use. It also helps control the rate of evaporation and the tackiness of the compositions.

The pH of the compositions of the present invention may be adjusted by one or more pH adjusters and/or chelating agents. For example, sodium hydroxide, triethanolamine, EDTA salt, or any combinations thereof, are suitable pH adjusters/chelating agents that may be included in the compositions of the present invention.

An effective amount of a pH adjuster and/or chelating agent is included to adjust the pH of the final composition to about 3 to about 9. Preferably, the pH is adjusted to about 5 to about 8 and more preferably about 6 to about 7.

One or more humectants may be used in the compositions of the present invention. Suitable humectants include, but are not limited to, glycerin, pentylene glycol, hexylene glycol, propylene glycol, butylene glycol, sorbitol, or any combinations thereof. One or more humectants may be included in the compositions of the present invention in an amount about 0.1 wt. % to about 15 wt. % of the total weight of the composition. Preferably, humectant is present in an amount about 1 wt. % to about 5 wt. % of the total weight of the composition.

Another component that may be used in the compositions of the present invention is a film former. The film former is a hydrophobic material that imparts film forming and waterproofing characteristics to the emulsion. Suitable film former for use in the compositions of the present invention include, but is not limited to, hydrogenated oils and esters, synthetic wax, natural wax, polypropylene, polyurethane, hydrogenated oils and esters, acrylates/acrylamide copolymer, acrylates copolymer, polyethylene, VP/dimethiconylacrylate/polycarbamylpolyglycol ester, butylated PVP, PVP/hexadecene copolymer, octadecene/MA copolymer, PVP/eicosene copolymer, tricontanyl PVP, Brassica Campestris/Aleuritis Fordi Oil copolymer, decamethyl cyclopentasiloxane (and) trimethylsiloxysilicate, or any combinations thereof. One or more film formers may be present in the compositions of the present invention in an amount about 0.1 wt. % to about 5 wt. % of the total weight of the composition. Preferably, the one or more film formers or waterproofing agents is present in the compositions of the present invention in an amount about 1 wt. % to about 3 wt. % of the total weight of the composition.

One or more preservatives may be included in the compositions of the present invention. The preservative protects the compositions from microbial contamination and/or oxidation. As such, the preservative can include an antioxidant. Preservatives can include but are not limited to, phenoxyethanol, methylparaben, ethylparaben, propylparaben, isobutylparaben and butylparaben, potassium sorbate, sorbic acid, ethyl hexyl glycerin, diazolidinyl urea, iodopropynyl butylcarbamate, chloromethylisotiazolinone, methylisothiazolinone, vitamin E and its derivatives including vitamin E acetate, vitamin C, butylated hydroxytoluene, butylparaben, ethylparaben, methylparaben, propylparaben, isobutylparaben, phenoxyethanol, or any combinations thereof, may be included as a preservative in a composition of the present invention.

About 0.01 wt. % to about 2 wt. % of preservative may be included in a composition of the present invention. Preferably, one or more preservatives total about 0.5 wt. % to about 1.5 wt. % of the total weight of the composition.

The compositions of the present invention may also have other optional additives including bio-active (functional) ingredients. For instance, one or more plant extracts, fruit extracts, vegetable extracts, algae extracts, sugars, polysaccharides, lipids, proteins, peptides, aminoacids, aminoacid derivatives, absorbents, salicylic acid, alpha and beta hydroxy acids, oil and water soluble vitamins including vitamins A, C, and E and their derivatives, or any combinations thereof, may be included in the compositions. When present, the optional additives may be included in the present composition in an amount about 0.001 wt. % to about 10 wt, %, based on the total weight of the composition.

In one embodiment, the composition may also comprise at least one coloring agent chosen from pigments, natural colorant, and dyes or a combination of pigments, natural colorants, and dyes. As used herein, pigments refer to colored solid particles at 25° C. that are not soluble in the liquid fatty phase. Pigments may include nacreous pigments (i.e., nacres), and pearling agents.

The at least one coloring agent may be chosen, for example, in order to obtain make-up compositions which give good coverage, in other words, which do not leave a significant amount of the at least one keratin material to which it is applied showing through. The coloring agent may also reduce the sticky feel of the compositions.

Representative oil soluble dyes which may be used include, but are not limited to, Sudan red, DC Red 17, DC Green 6, β-carotene, soybean oil, Sudan brown, DC Yellow 11, DC Violet 2, DC Orange 5, annatto, and quinoline yellow and natural oil soluble dyes. The liposoluble dyes, when present, may have a concentration ranging up to 20% by weight of the total weight of the composition, such as from 0.01% to 6%.

Representative water soluble dyes which may be used include, but are not limited to DC red 33, red 22, red 28, blue 1, yellow 5, yellow 6, red 40, yellow 10, green 3, green 5, green 8 and natural water soluble dyes. The water soluble dyes, when present, may have a concentration ranging up to 20% by weight of the total weight of the composition, such as from 0.01% to 6%.

In one aspect, the pigments may be chosen from white, colored, mineral, organic, coated and uncoated pigments and encapsulated pigments. Representative examples of mineral pigments include, but are not limited to, titanium dioxide, which may be optionally surface-treated, zirconium oxide, zinc oxide, cerium oxide, iron oxides, chromium oxides, manganese violet, ultramarine blue, chromium hydrate and ferric blue. Representative examples of organic pigments include, but are not limited to, carbon black, pigments of D & C type, and lakes based on cochineal carmine, barium, strontium, calcium and aluminum. If present, the pigments may have a concentration ranging up to 40% by weight of the total weight of the composition, and for example from 1% to 35%, and further such as from 2% to 25%. In one embodiment comprising a face powder product, the pigments, including nacreous pigments, may, for example, represent up to 50% by weight of the composition.

In one aspect, the nacreous pigments (or nacres) may be chosen from, but are not limited to, white nacreous pigments such as mica coated with titanium or with bismuth oxychloride; colored nacreous pigments such as titanium mica with iron oxides, titanium mica with ferric blue or chromium oxide, and titanium mica with an organic pigment chosen from those mentioned above; and nacreous pigments based on bismuth oxychloride. The nacres, if present, may have a concentration ranging up to 30% by weight of the total weight of the composition, such as from 0.1% to 20%.

Pigments also include a blend of iron oxides encapsulated with a material such as a polymer, a pigment, a wax, a sugar derivative, or a combination of the proceeding material. Encapsulation of pigments are disclosed in patent application U.S. 20110229536 which processes and definitions are hereby incorporated by reference.

In one aspect, the composition may include fillers including, but not limited to, talc, kaolin, silica, barium sulfate, aluminum hydroxide, calcium silicate, silica, silicone powders, and acrylic acid copolymers.

In addition, the compositions may include one or more of the following components: sunscreen agent, SPF booster, secondary emulsifier, emollient, moisturizer, humectant, film former/waterproofing agent, bio-active (functional) ingredient, fragrance, or any combinations thereof.

It is, of course, understood that the present cosmetic composition may further include optional ingredients generally known by one skilled the art to be suitable for use in a cosmetic composition. Lists of carriers and optional ingredients, which are well known in the art, are disclosed, for example, in “Cosmetics: Science and Technology,” edited by M. S. Balsam and E. Sagarin, 2nd Edition, 1972, Wiley Pub. Co.; “The Chemistry and Manufacture of Cosmetics” by M. G. DeNavasse; and “Harry's Cosmeticology,” J. B. Wilkinson et al., 7th Edition, 1982, Chem. Pub. Co.; the disclosures of each of the above being incorporated herein by reference.

The composition may be in a variety of forms of make-up composition applied to keratinous surfaces for the purpose of coloring, conditioning, or otherwise beautifying the keratinous surface. Make-up is also known as color cosmetics and includes products such as eye shadow, blush, concealer, lipstick, lip gloss, or eyeliner compositions in a solid, or stick form.

The examples below illustrate exemplary formulations of the cosmetic composition. They are not intended as a limitation upon the scope of the present application.

### EXAMPLE 1

Compositions tested contained PEG/PPG emulsifier, diluent, emollient, and water.

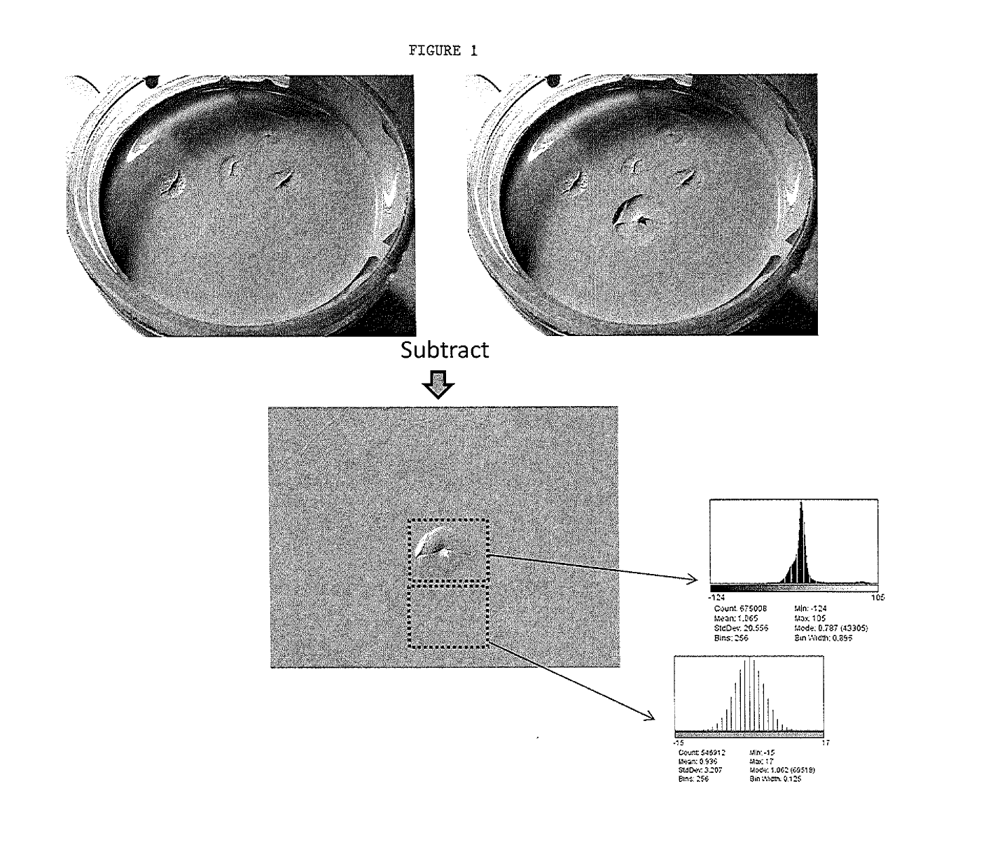

The variation of configuration from an initial configuration having a bin width of about 0.15 to a deviation from the initial configuration to a bin width of about 0.9 after application of about 0.5 kPa to about 5 kPa of pressure to the composition is depicted in FIG. 1.

Bin width is a measure of pixel light intensity variation (Lmax and Lmin). Lmax represents light intensity value for the lightest pixel in a photograph. Lₘᵢₙ represents the light intensity value for the darkest pixel in a photograph. For 8-bit images, the light intensity values can vary from 0 to 255. The bin width for the image would be maximum lightness variation (Lmax-Lmin) divided by number of bins (256 for an 8-bit gray scale image) according to the following equation:

$$Bin Width = ( \frac{{L}_{max} - {L}_{min}}{Number of Bins} ) {}_{8 Bit gray scale image} ( \frac{{L}_{max} - {L}_{min}}{256} )$$

The experiment was repeated with samples having various concentration ranges of PEG/PPG emulsifier, diluent, emollient, and water with a range in pressure from about 0.5 to about 5 KPa. The initial bin width was 0.05 to about 0.15 range. The disturbed surface bin width was 0.8 to 1.0 range. Compositions returned to the initial bin width (configuration) within 2 to 4 hours resting undisturbed. The bin width for the image would be maximum lightness variation (Lₘₐₓ-Lₘᵢₙ) divided by number of bins (256 for a 8-bit gray scale image) according to the above equation.

In an example a composition in an initial configuration was exposed to a defused light source. An image was taken of the composition at a forty-five degree angle to the surface of the composition. The surface of the composition was disturbed by application of about 0.5 to about 5 kPa of pressure with the finger or any instrument or applicator. Without change in the light source, the composition position, or camera position, an image was taken of the surface of the composition that deviates from the initial configuration (reference image). The composition was allowed to sit undisturbed and images were taken every fifteen minutes until the composition returned to the initial configuration. The time it took to return to the initial configuration after application of pressure to the surface was about 4 hours. The change in surface was measured by subtracting the reference image from the image taken at any given time. The images were converted into an 8-bit gray scale image using http://rsbweb.nih.gov/ij/ software. The disturbed areas caused by the finger (or applicator) in each subtraction image were individually analyzed using Image J software (hftp://rsbweb.nih.gov/ij/) and bin width values were measured. The initial configuration had a bin width of 0.15. The disturbed surface (surface that deviates from an initial configuration) had a bin width of 0.9. After four hours the composition returned to a bin width value of 0.15.

The amount of average pressure exerted on a cosmetic composition while touching it with a finger (application of pressure), was measured as follows:
a. The cosmetic composition was placed on a scale. A volunteer touched the composition surface lightly with an index finger.
b. The surface area of contact by the finger was calculated using the Image J software (http://rsbweb.nih.gov/ij/). The distances for the measurements were calibrated by a ruler in the picture.
c. The force (a)/surface area (b)=pressure.
d. The aforementioned procedure was repeated at least 5 times for each person and in total 6 people participated in this part of study. The pressure values ranged from 0.6 to 4.8 KPa.

### EXAMPLE 2

| Seq | Raw Material | Exp 1 | Exp 2 | Exp 3 | Exp 4 | Exp 5 | Exp 6 |
|---|---|---|---|---|---|---|---|
| 1 | PEG/PPG 19/19 | 10.59% | 10.59% | 10.59% | 10.59% | 10.59% | 10.59% |
| | Dimethicone* | | | | | | |
| 1 | C 13-16 | 10.59% | 10.59% | 10.59% | 10.59% | 10.59% | 10.59% |
| | Isoparaffin, | | | | | | |
| | C10-13 | | | | | | |
| | Isoparaffin* | | | | | | |
| 2 | 1.8 cst | 77.82% | 73.82% | 68.82% | 63.82% | 58.82% | 53.82% |
| | dimethicone* | | | | | | |
| 3 | Water | 1.00% | 5.00% | 10.00% | 15.00% | 20.00% | 25.00% |
| 3 | Butylene Glycol | | | | | | |
| 3 | Glycerin | | | | | | |
| | Total | 100.00% | 100.00% | 100.00% | 100.00% | 100.00% | 100.00% |
| | Viscosity (cps) | 7,000 | 25,600 | 47,000 | 63,000 | 82,000 | 103,000 |

| Seq | Raw Material | Exp 7 | Exp 8 | Exp 9 | Exp 10 | Exp 11 |
|---|---|---|---|---|---|---|
| 1 | PEG/PPG 19/19 | 10.59% | 10.59% | 10.59% | 10.59% | 10.59% |
| | Dimethicone* | | | | | |
| 1 | C 13-16 | 10.59% | 10.59% | 10.59% | 10.59% | 10.59% |
| | Isoparaffin, | | | | | |
| | C10-13 | | | | | |
| | Isoparaffin* | | | | | |
| 2 | 1.8 cst | 48.82% | 38.82% | 28.82% | 61.72% | 61.72% |
| | dimethicone* | | | | | |
| 3 | Water | 30.00% | 40.00% | 50.00% | | |
| 3 | Butylene Glycol | | | | 17.10% | |
| 3 | Glycerin | | | | | 17.10% |
| | Total | 100.00% | 100.00% | 100.00% | 100.00% | 100.00% |
| | Viscosity (cps) | 137,000 | 184,000 | 570,000 | | |

Sequence 1 was added to Sequence 2 in a beaker and heated to 70° C., while mixing with a propeller. Sequence 3 water was added to beaker slowly while mixing. Batch was mixed for 2 minutes before dropping into jars. 200 g batches were prepared in this manner.

Exp 1: Batch did not seem to increase in viscosity when water was added, and remained clear during mixing. Batch formed a clear, slightly viscous liquid with a very soft elastic gel-like structure after cooling. 24 Hour viscosity RVTA@5 rpm 17.5, 7,000 cps.

Exp2: Batch increased in viscosity slightly when water was added, and became translucent during mixing. Batch formed a slightly translucent very soft recovering elastic gel structure after cooling, 24 Hour viscosity RVTB@5 rpm 32.0, 25,600 cps.

Exp 3: Batch increased in viscosity slightly when water was added, and became opaque during mixing. Batch formed a slightly soft recovering elastic gel structure after cooling. 24 Hour viscosity RVTC@5 rpm 23.5, 47,000 cps.

Exp 4: Batch increased in viscosity slightly when water was added, and became opaque during mixing. Batch formed a slightly soft recovering gel structure after cooling. 24 Hour viscosity RVTC@5 rpm 31.5 , 63,000 cps.

Exp 5: Batch increased in viscosity when water was added, and became opaque during mixing. Batch formed an elastic recovering gel structure after cooling. 24 Hour viscosity RVTC@5 rpm 41.0, 82,000 cps.

Exp 6: Batch increased in viscosity when water was added, and became opaque during mixing. Batch formed a more firm elastic gel structure after cooling, that does not seem to recover as quickly after disturbance. 24 Hour viscosity RVTC@5 rpm 51.5, 103,000 cps.

Exp 7: Batch increased in viscosity when water was added, and became opaque during mixing. Batch formed a firm elastic gel structure after cooling, that does not seem to recover quickly after disturbance. 24 Hour viscosity RVTC@5 rpm 68.5, 137,000 cps.

Exp 8: Batch increased in viscosity when water was added, and became opaque during mixing. Batch formed a firm elastic gel structure after cooling, that doe s not seem to recover after disturbance, and breaks apart easily. 24 Hour viscosity RVTD@5 rpm 46.0, 184,000 cps.

Exp 9: Batch increased in viscosity when water was added, and became opaque during mixing. Batch formed a rigid gel structure after cooling, that doe s not seem to recover after disturbance, and breaks apart very easily. 24 Hour viscosity RVTE@5 rpm 57.0, 570,000 cps.

Exp 10: Batch did not increase in viscosity when butylene glycol was added, became opaque during mixing, and did not form a gel after cooling.

Exp 11: Batch did not increase in viscosity when glycerin was added, became translucent during mixing, and did not form a gel after cooling.

### EXAMPLE 3

| Seq | Raw Material | Exp 1 | Exp 2 | Exp 3 | Exp 4 | Exp 5 | Exp 6 | Exp 7 | Exp 8 | Exp 9 | Exp 10 |
|---|---|---|---|---|---|---|---|---|---|---|---|
| 1 | PEG/PPG 19/19 | 10.59% | 10.59% | 10.59% | 10.59% | 10.59% | 10.59% | 10.59% | 10.59% | 10.59% | 10.59% |
| | Dimethicone* | | | | | | | | | | |
| 1 | C 13-16 | 10.59% | 10.59% | 10.59% | 10.59% | 10.59% | 10.59% | 10.59% | 10.59% | 10.59% | 10.59% |
| | Isoparaffin, | | | | | | | | | | |
| | C10-13 | | | | | | | | | | |
| | Isoparaffin* | | | | | | | | | | |
| 2 | 1.8 cst | 61.72% | | | | | | | | | |
| | dimethicone | | | | | | | | | | |
| 2 | 5 cst | | 61.72% | | | | | | | | |
| | dimethicone | | | | | | | | | | |
| 2 | 10 cst | | | 61.72% | | | | | | | |
| | dimethicone | | | | | | | | | | |
| 2 | 100 cst | | | | 61.72% | | | | | | |
| | dimethicone | | | | | | | | | | |
| 2 | Cyclopentasiloxane | | | | | 61.72% | | | | | |
| 2 | Isodocecane | | | | | | 61.72% | | | | |
| 2 | Neopentyl Glycol | | | | | | | 61.72% | | | |
| | Diheptanoate | | | | | | | | | | |
| 2 | Isotridecyl | | | | | | | | 61.72% | | |
| | Isononanoate | | | | | | | | | | |
| 2 | Pentaerythrityl | | | | | | | | | 61.72% | |
| | Tetraisostearate | | | | | | | | | | |
| 2 | Triisostearyl | | | | | | | | | | 61.72% |
| | Citrate | | | | | | | | | | |
| 3 | Water | 17.10% | 17.10% | 17.10% | 17.10% | 17.10% | 17.10% | 17.10% | 17.10% | 17.10% | 17.10% |
| | Total | 100.00% | 100.00% | 100.00% | 100.00% | 100.00% | 100.00% | 100.00% | 100.00% | 100.00% | 100.00% |

*BY 25-337 from Dow Corning: PEG/PPG 19/19 dimethicone, C 13-16 Isoparaffin, C10-13 Isoparaffin

Sequence 1 was added to Sequence 2 in a beaker and heated to 70° C., while mixing with a propeller. Sequence 3 water was added to beaker slowly while mixing. Batch was mixed for 2 minutes before dropping into jars. 200 g batches were prepared in this manner.

EXP 1: Seq 1 and Seq 2 appeared clear when mixed together. When water was added batch became opaque and thickened. Batch thinned after 2 minutes of mixing. Batch had a recovering elastic gel structure after cooling. 24 hour viscosity RVTC@5 rpm DR 35.5, 71,000 cps.

EXP 2: Seq 1 and Seq 2 appeared clear when mixed together. When water was added batch became opaque and thickened. Batch thinned out after 2 minutes of mixing. Batch had a recovering gel structure after cooling. 24 hour viscosity RVTC@5 rpm DR 28.0, 56,000 cps.

EXP 3: Seq 1 and Seq 2 appeared clear when mixed together. When water was added batch became opaque and thickened. Batch thinned out after 2 minutes of mixing. Batch had a slightly soft recovering gel structure. 24 hour viscosity RVTC@5 rpm DR 20.5, 41,000 cps.

EXP 4: Seq 1 and Seq 2 became cloudy when mixed together. Batch did not thicken when water was added. Batch did not form a recovering elastic gel structure when cooled and began to separate soon after dropping.

EXP 5: Seq 1 and Seq 2 appeared clear when mixed together. When water was added batch became opaque and very viscous. Batch thinned slightly while mixing. Batch was more viscous than the other batches when dropped, and formed a more firm recovering gel structure than the other batches. 24 hour viscosity: RVTC@5 rpm DR 60.0, 120,000 cps.

EXP 8: Seq 1 and Seq 2 appeared clear while mixing, became only slightly translucent when water was added, but became opaque while mixing. Batch was thin when dropped, but did form a soft recovering elastic gel structure when cooled. 24 hour viscosity: RVTC@5 rpm DR 17, 34,000 cps.

EXP 7: Seq 1 and Seq 2 appeared clear while mixing, became opaque when water was added, and became foamy while mixing. Batch did not for any gel structure, and was liquid.

EXP 8: Seq 1 and Seq 2 appeared clear while mixing. Became opaque when water was added, and became foamy while mixing. Batch formed a loose cream, and began separating after less than 24 hours.

EXP 9:Seq 1 and Seq 2 became cloudy when mixed together. Batch became opaque when water was added and took on a grainy appearance while mixing. After dropping batch solids separated out. No gel structure was formed.

EXP 10: Seq 1 and Seq 2 became cloudy when mixed together. Batch became opaque when water was added and took on a grainy appearance while mixing. After cooling, batch separated into 2 layers, the top layer was a white, firm, elastic gel. A uniform gel structure was not formed.

### EXAMPLE 4

Use of a different diluent in this system

| Seq | Raw Material | Exp 1 |
|---|---|---|
| 1 | PEG/PPG 19/19 Dimethicone* | 10.59% |
| 1 | Cyclopentasiloxane* | 10.59% |
| 2 | 1.8 cst dimethicone | 61.72% |
| 3 | Water | 17.10% |
| | Total | 100.00% |

*BY 11-030 from Dow Corning: Cyclopentasiloxane, PEG/PPG 19/19 dimethicone

1. A composition comprising an about 7.5% to about 11.0% PEG/PPG emulsifier, an at least one diluent, an at least one emollient, and <25 weight % water, wherein the composition deviates from an initial configuration after application of about 0.5 to about 5 kPa of pressure on the composition and returns to the initial configuration in less than about 4 hours at room temperature.

2. The composition of claim 1, wherein the emollient comprises a straight chain hydrocarbon.

3. The composition of claim 1, wherein the emollient comprises a cyclic hydrocarbon.

4. The composition of claim 2, wherein the emollient comprises <100 cst dimethicone.

5. The composition of claim 1, wherein the composition is in the form of a make-up.

6. The composition of claim 5, wherein the make-up is an eye shadow, a concealer, an eyeliner, a make-up for the lips, a make-up for the eyelashes, a make-up for the eyebrows, and a make-up for the face.

7. The composition of claim 1, wherein the PEG/PPG emulsifier is PEG/PPG 19/19 dimethicone.

8. The composition of claim 1, wherein the dimethicone is present in an amount of about 60.0 weight %.

9. The composition of claim 1, wherein said PEG/PPG emulsifier is PEG/PPG 18/18 dimethicone.

10. The composition of claim 1, wherein the composition further comprises a non-polar solvent.

11. The composition of claim 1, wherein the composition includes optional ingredients such as a coloring agent chosen from a dye, a natural colorant, a pigment or a combination of a dye, a natural colorant, and a pigment.

12. The composition of claim 1, wherein the composition includes one or more optional ingredients comprising sunscreen agent, secondary emulsifier, secondary emollient, moisturizer, humectant, film former, bio-active (functional) ingredient, pH adjuster/chelating agent, preservative, fragrance, or any combinations thereof.

13. The composition of claim 1, wherein the composition has a stable viscosity.

14. The composition of claim 1, wherein the diluent is isoparaffin.

15. The composition of claim 1, wherein the diluent is cyclopentasiloxane.

16. The composition of claim 1, wherein the water concentration is about 1.0 weight % to about 17.5 weight %.

17. The composition of claim 16, wherein the water concentration is about 17 weight %.

18. The composition of claim 13, wherein the stable viscosity is determined by measurement of the initial viscosity.

19. The composition of claim 18, wherein the initial viscosity is about 170,000 to about 670,000 CPS.
